(19) United States
(12) Patent Application Publication
(10) Pub. No.: US 2019/0008447 A1
(43) Pub. Date: Jan. 10, 2019

(54) DETECTION DEVICE FOR DETECTING AND MONITORING A BODY POSTURE OR A SEQUENCE OF MOVEMENTS OF A BODY PART AND METHOD THEREFORE

(71) Applicant: intrOnic GmbH & Co., Waiblingen (DE)

(72) Inventor: Michael Lacker, Waiblingen (DE)

(21) Appl. No.: 15/753,801

(22) PCT Filed: Aug. 16, 2016
(86) PCT No.: PCT/EP2016/069449

§ 371 Date: Feb. 20, 2018

(30) Foreign Application Priority Data
Aug. 20, 2015 (DE) 10 2015 113 816.6

(51) Int. Cl. A61B 5/00; A61B 5/11

(52) U.S. Cl. CPC A61B 5/4561; A61B 5/1116; A61B 5/742; A61B 5/0077; A61B 2562/0261

(57) ABSTRACT

The invention relates to a device for detecting and monitoring posture or a sequence of movements or a training sequence of a body part. A support element (12) which comprises an adhesive layer (14) on one side for adhering to the skin of a user is provided, said support element comprising a strain measuring device (16), the measurement signals in at least one direction of extension of the support element (12) being detected by an elongation or compression of the support element (12) and is forwarded to a circuit (17) which is in contact with the strain measuring device (16). The invention also relates to a method therefore (see FIG. 7).

The invention relates to a detection device for detecting and monitoring a posture or a movement sequence of a body part, and a method for recognising and monitoring for this.

A monitoring, correcting and conditioning device for a posture of a person is known from DE 20 2012 005 018 U1. This system comprises one or more cameras and a piece of data processing software. This comprises a piece of posture recognition software. The images detected by means of the camera by the person to be monitored are evaluated by this posture recognition software in order to check whether the optimum ergonomic posture is present or is maintained when working at a desk or a screen. In the event of deviations from the optimum ergonomic posture, the programme gives correction instructions and tips for prevention and self-treatment.

This monitoring, correcting and conditioning device for posture is linked exclusively to use in the workplace and is very laborious in terms of technology.

A device for monitoring the posture and/or movement of a human body part is known from DE 20 2005 015 889 U1. Here, is it provided that the device for monitoring the posture and/or movement of the human body has at least one sensor that is mounted on the monitoring body part. For example, this sensor, which can be formed as stretch measuring strips, can be fixed on a bandage or a corresponding piece of clothing in order to position it on the body.

Furthermore, a device for detecting parameters for characterising movement sequences on the human body is known from DE 10 2008 052 406 A1, said device comprising a bending sensor. This fixing sensor comprises a fixing plaster for fixing the bending sensor on the skin of the human body. This fixing element comprises a cavity in which a bending sensitive detector is inserted. This detector 10 consists of a tape or strip-like substrate which is formed from spring steel. Stretch strips that are each aligned opposite one another are arranged on opposing sides of the substrate. This plurality of stretch strips detects a mechanical shaping of the substrate.

The object of the invention is to propose a detection device and a detection unit for detecting and monitoring a posture or a movement sequence of a body part, in particular adopting a predetermined position or carrying out a movement or training sequence of the body part, which enables a use that is independent and not connected to a location, in particular not to a workplace.

The object of the invention is furthermore to propose a method for recognising and monitoring a posture or a movement sequence of a body part that signals to the user the change of an optimum posture or movement sequence, even in the event of minor deviations.

This object is solved by means of a detection device having a carrier element which, on one side, comprises an adhesive later for adhering the carrier element to a skin of the user, and a stretch measuring device is provided on or in the carrier material, which detects measuring signals in at least one extension device of the carrier element by a stretch or compression of the carrier element, and has a circuit formed as a transponder that is contacted with the stretch measuring device and that the circuit is fixed on the carrier element. Such a detection device enables this to be able to be applied directly to the skin of the user in a simple manner, and thus minor changes in terms of posture or a movement sequence can already be detected. In addition, this detection device has the advantage that it can be worn under conventional clothing without it being outwardly visible or noticeable. Furthermore, such a detection device can be used on different body regions. In addition, the user is not linked to a location when using such a detection device.

It is preferably provided that the carrier material with the stretch measuring device and the circuit is formed as a disposable product. Such a detection device is intended for single use. This detection device is adhered to the concerned points of the body of the user. After the desired monitoring time period has finished or an adhesive effect of the adhesive material on the carrier device loses its effectivity, this detection device is thrown away. Then, in the event of a new monitoring, the use of a new detection device is provided.

Furthermore, it is preferably provided that the carrier element is formed in the shape of a tape. Preferably, an elongated strip-like material is provided for producing the carrier element, which is preferably provided ready-for-use in different lengths and/or widths. Depending on the usage situation, long strip-like detection devices can be used, for example, along the spinal column. Furthermore, short strip-like carrier elements can be used, for example, in the throat or neck region.

Preferably, the carrier element is formed as a woven tape, a breathable and/or air-permeable film, as fleece or felt. Thus, the detection device is flexible in adjusting to different body contours and body regions. So-called tapes are preferably used, as they are known from kinesiology.

Preferably, the carrier element contains a storage element and/or an acceleration sensor. This storage element can be integrated into the circuit.

A further preferred embodiment of the detection device provides that at least the stretch measuring device and/or the circuit and/or the storage element and/or the acceleration sensor are applied on the carrier element. For example, these components can be adhered to, pressed on, knife-coated, moulded on and/or even sprayed onto the carrier element. As a result, a construction with a low layer thickness and a high flexibility for adjusting to body contours is made possible.

Advantageously, at least the stretch measuring device and/or the circuit and/or the storage element that are applied to the carrier element are covered by a protective film. As a result, these are protected from premature damage.

A further alternative embodiment of the detection device provides that at least the stretch measuring device is woven into the carrier material, and preferably, at least the circuit and/or the storage element are arranged on one of the two sides of the carrier element or inserted into an indent of the carrier element. As a result, as early as during the production of the carrier element, an integration of the stretch measuring device is made possible.

An alternative embodiment of the detection device provides at least the stretch measuring device and/or the circuit and/or the storage element and/or the acceleration sensor at least partially between the carrier element and an adhesive layer applied thereon. As a result, a simple integration and still a direct positioning of the stretch measuring device on the skin can be made possible.

The stretch measuring device applied to the carrier element comprises at least one stretch measuring strip or at least one wire element that detects a compression and/or stretch that is transferred to the carrier element by the user in the event of a change of his posture or a movement sequence. Preferably, two or more stretch measuring strips or wire elements can be used for forming the stretch measuring device. These can each be provided separately or spatially detached from one another on the carrier element and point in directions deviating from one another. Preferably, three stretch measuring strips lying one on top of the other are provided as the stretch measuring device that are formed in the shape of a stretch measuring rosette, i.e. these are aligned relative to one another at an angle deviation of 45° in each case. In particular, the stretch measuring strips lying one over the other can be separated from one another by means of a dielectric material. The stretch measuring strips and the dielectric material arranged therebetween can be applied to the carrier element one after the other by means of pressure technology.

Furthermore, it is preferably provided that the at least one stretch measuring strip or the at least one wire element is provided pre-tensioned on the carrier element. As a result, both a compression and a stretch of the carrier element can be detected in equal measure by corresponding body movements, such as bending the back or adopting an upright position, for example.

Furthermore, the stretch measuring device consisting of several stretch measuring strips or wire elements preferably has at least one stretch measuring strip that is woven into or stitched onto the carrier material and/or comprises at least one stretch measuring device and/or at least one wire element that is applied to the carrier element, in particular stitched on, stitched in or adhered on. Here, these can be provided on the carrier element to be aligned in the same or different directions. The wire element can be woven in as a warp and/or weft thread. The wire element can also be introduced into the weave retrospectively by threading.

The circuit of the detection device can, furthermore, comprise an electronic control system and a data transfer device. As a result, the signals detected by the stretch measuring device and stored in the storage element can be read.

Furthermore, the detection device can preferably comprise a current accumulator connected to the circuit. This can take place, for example, in the form of a capacitor, a film battery or a lithium polymer. As a result, the requests for a construction smaller in height or for integration into the carrier element can be fulfilled.

According to a further advantageous embodiment of the detection device, the carrier material can receive an acoustic and/or vibration element. As a result, in the event of a change of the posture to be monitored or the movement sequence to be monitored, an acoustic and/or vibrating signal can be emitted immediately, which immediately indicates the detected change to the user, such that he can immediately undertake a correction.

Furthermore, according to a further alternative embodiment of the detection device, an activation button can be provided on or in the carrier element, by means of which activation button the circuit can be activated. As a result, the user can start the detection and monitoring of the posture on the carrier element himself after applying the detection device to the skin and optionally also end it again at a desired point in time.

Alternatively, the circuit can be immediately activated by a mobile terminal device. Here, a contactless data transfer between the circuit and the mobile terminal device is provided. Furthermore, the mobile terminal device can alternatively communicate with the activation button that, in turn, activates the circuit. In this embodiment, the activation of the circuit can take place both by the user using the activation button directly or by a mobile terminal device.

An alternative embodiment of the detection device provides that the circuit is formed as a passive or active transponder, and a data transfer device for the contactless data transfer between a mobile terminal device or a reading device and the transponder is provided. For example, the dielectric components can be formed to form a passive transponder which enables the stored data to be read by a reading device after introduction into an electromagnetic alternating field. In the further embodiment, at least one signal is given to the user as an active transponder via the mobile terminal device, that a change in the posture in comparison to the desired or optimum posture has taken place or there has been a deviation in the movement sequence to be monitored. Here, the signals can be prepared and emitted in a different manner on the mobile terminal device. Acoustic signals, vibrations and/or optical signals can be emitted by the display of the mobile terminal device.

Preferably, a removable protective film is applied to the adhesive layer of the carrier element. As a result, the detection device can be provided for use directly after removing this protective film and can be used.

Furthermore, a detection unit is provided according to the invention that wirelessly communicates according to an embodiment described above from a mobile terminal device having a data processing programme stored thereon and a detection device. Alternatively, the detection unit can comprise a data processing programme (app) that can be downloaded onto a mobile terminal device and a detection device according to one of the preceding embodiments that wirelessly communicate with each other.

The object underlying the invention is furthermore solved by a method for detecting and monitoring a posture or a movement sequence or a training sequence of a body part, wherein a carrier element that has a stretch measuring device and a circuit contacted therewith is applied to a skin of a user by means of the adhesive layer and wherein a calibration routine is started by an activation button received by the carrier element and connected to the circuit and/or by an input on the mobile terminal device, and, after the calibration routine has taken place, the recognition and monitoring of the posture or a movement sequence or the training sequence of a body part is started and the change in the posture or the movement sequence to be monitored or the training sequence is detected by the circuit and at least one signal is emitted to the user as an indication of the detected change in posture or movement sequence or training sequence of the body part. According to a first embodiment, the detection device can be started by the activation button provided on the carrier element in a simple manner and independent of further components and taken into operation. According to an alternative embodiment according to the invention, the method can also be started by the mobile terminal device.

When using a detection device in which the carrier element receives at least one acoustic and/or vibration element, the deviation in posture or movement sequence or training sequence of the body part can be emitted by an acoustic and/or vibration element based on the original dataset by the calibration routine. As a result, the user can be notified of a changed posture or a change in the movement sequence without carrying further mobile terminal devices.

Alternatively, the signal that signals a change in the posture or the movement sequence or the training sequence relative to the original dataset can be contactlessly transmitted to a watch, a tablet, mobile telephone, reading device or similar by means of a data transmission. The selection to emit the transmitted signal through the mobile terminal device can be selectively set by the user, such that this obtains an acoustic, optical and/or vibrating signal.

Furthermore, the data transmitted to the mobile terminal device and received by this are stored in an app and emitted. As a result, a recording, for example via the carrying duration and adherence of the ergonomically favourable posture and/or the movement sequence to be monitored or training sequence of a body part, can take place. These data stored therein can, for example, be forwarded to health insurance companies or Employers' Liability Insurance Associations which reward the user by means of a bonus system or similar with regular use of the detection devices. In addition, the user himself can start a query at any time and monitor himself.

An advantageous embodiment of the method provides that a change of the posture in the monitoring phase is monitored by an acceleration sensor, and preferably a change of the posture to be monitored that takes place only momentarily and returns to the posture to be monitored is recognised and not emitted as an alarm signal. As a result of such an acceleration sensor, a starting position or the posture to be monitored is detected. As soon as a movement outside this position takes place and returns to the previously stored posture or the posture to be monitored within a predetermined duration of time, this is recognised by means of the acceleration sensor, since, in turn, the starting position or the position to be monitored has been adopted. In such cases, an indication to the user in the form of a signal or an alarm signal is not emitted. An exemplary usage for this is that, with a person sitting at a desk, the sitting position is monitored. If this person reaches for the telephone receiver, a position that deviates from the position to be monitored is adopted, but since the posture to be monitored is, however, adopted again after putting down the receiver, the acceleration sensor recognises this. Such a momentary change is thus not interpreted as an incorrect posture and correspondingly not displayed. However, as soon as the person stands up, a change of the body position to be monitored, i.e. seating position, is detected, such that either the monitoring phase is interrupted or an alarm signal is emitted. This example can be transferred to further different usages.

As a result of such a method, for example, a posture with a person sitting or standing, in particular with regularly repeating work, can be monitored in a simple manner. Activities to be carried out regularly, such as assembly work by a worker or repetitive movement sequences, for example, can also be monitored. The monitoring of the posture or the movement sequence can also make is possible for a person to adopt certain postures at regular time intervals that, in particular, can be freely set, said postures being recommended for relaxing and stretching in order to counteract the continuous adoption of an incorrect body posture or body position. Furthermore, this method for monitoring a training sequence can be used, in particular, for back and spine exercise classes. The user can be asked, after a predetermined time duration, for example by an optical, acoustic and/or vibrating signal, to carry out a corresponding movement sequence or adopt a certain posture in order to relax and/or stretch individual body parts, in particular. After adopting this posture or carrying out the movement sequence, this is detected and stored. As soon as the user carries out and also fulfills these tasks at the predetermined intervals, a signal, a confirmation or a reward, for example a smiley, can be emitted or bonus points for this are awarded and collected that can then be correspondingly redeemed. For example, health insurance companies can provide a fee reduction or issue vouchers.

In particular, it can be provided that a conditioning of the user in terms of one or more postures to be adopted is made possible as a result of the monitoring of the posture of the user. As a result of such a conditioning, engaging the cerebrum to adopt a conscious posture can be avoided, whereby the aim is achieved that a reaction is intuitively triggered in the user in order to adopt the correct posture or desired postures himself or carry out the movement sequences. As a result of interval monitoring of the posture or postures, this can also be trained again and again and further conditioned.

With a detection device having an acoustic element and/or vibration element, a calibration routine is introduced preferably by the activation button on the carrier element or by the mobile terminal device, in which calibration routine a first acoustic and/or vibration signal is emitted, and the user adopts the posture to be monitored. Subsequently, a confirmation signal for the detection of a so-called original dataset is emitted by the detection device after a predetermined time interval. Alternatively, this can be confirmed by pressing the activation button or by the mobile terminal device. The user then adopts a first incorrect posture. The resulting signal is detected and is confirmed by at least one second confirmation signal. Subsequently, a further incorrect posture can be adopted, the resulting signal detected and confirmed in turn. After the second or further confirmation signal of a first and, if applicable, second incorrect posture, the calibration routine is then ended, and the monitoring mode is started automatically. As a result, the detection device can be can be tuned to the respective place on the body to be monitored or accompanying posture directly after the application on the skin of the user within a very short time duration by this calibration routine, and started.

Preferably, with a detection device having an acoustic and/or vibration element, the calibration routine is started by an activation button provided on the carrier element or via the mobile terminal device in order to monitor a movement sequence or training sequence, and a first acoustic and/or vibrating signal is emitted, upon which the user adopts a starting position for the movement sequence to be monitored. Subsequently, after a predetermined time interval or by actuating the activation button or via the mobile terminal device, a confirmation signal is emitted for the detection of a so-called original dataset. Alternatively, this can be confirmed by pressing the activation button or via the mobile terminal device. The user carries out his movement sequence to be monitored, and the body part returns to the starting position. Subsequently, a further confirmation signal is emitted. This routine can be repeated once or several times. Subsequently, the calibration routine is ended, and the monitoring and/or training mode is started. As a result of this predetermined sequence of calibration steps, which is provided both for the monitoring of a movement sequence or training sequence and a posture, a simple and reliable operation and use of the detection device can be ensured.

With a detection device that communicates with a mobile terminal device, the calibration routine can be started by the mobile terminal device. The user adopts the posture to be monitored. After a predetermined time duration or after a confirmation by the user, this posture is detected and stored as an original dataset. Subsequently, the user adopts a first incorrect posture. The resulting signal is detected and at least one confirmation signal is emitted. Then, the calibration routine is ended, and the monitoring and/or training mode is started. With the communication to a mobile terminal device, the emission of the detected signals of the stretch measuring device can take place via the display, acoustically and/or by a vibration or any combination thereof.

With the use of the detection device having a mobile terminal device, the calibration routine for monitoring a movement sequence of a body part is, in turn, started by the mobile terminal device. The user brings the body part whose movement sequence is to be monitored into a starting position. Then, after a predetermined time period or by the confirmation of the user, this starting position is detected and stored as an original dataset. The user then carries out the movement sequence to be monitored and returns to the starting position with the body part. The resulting signals are detected and confirmed by at least one confirmation signal. Then, the calibration routine is ended, and the monitoring and/or training mode is started automatically.

The invention and further advantageous embodiments and developments of the same are described and explained in more detail below by means of the examples depicted in the drawings. The features that can be seen in the description and the drawings can be applied individually or in several different combinations according to the invention. Here are shown:

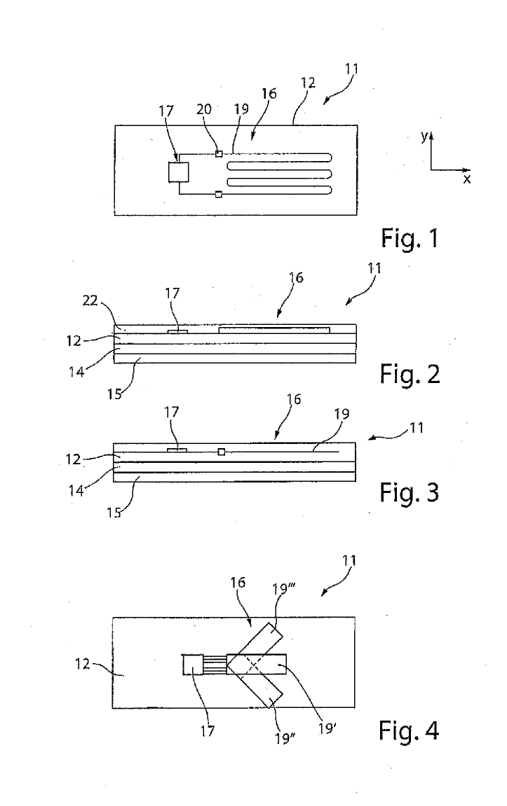

FIG. 1, a schematic view of the detection device according to the invention,

FIG. 2, a schematic side view of the detection device according to the invention according to FIG. 1,

FIG. 3, a schematic side view of an alternative embodiment of the detection device to FIG. 1,

FIG. 4, a schematic top view of a further alternative embodiment of the detection device to FIG. 1,

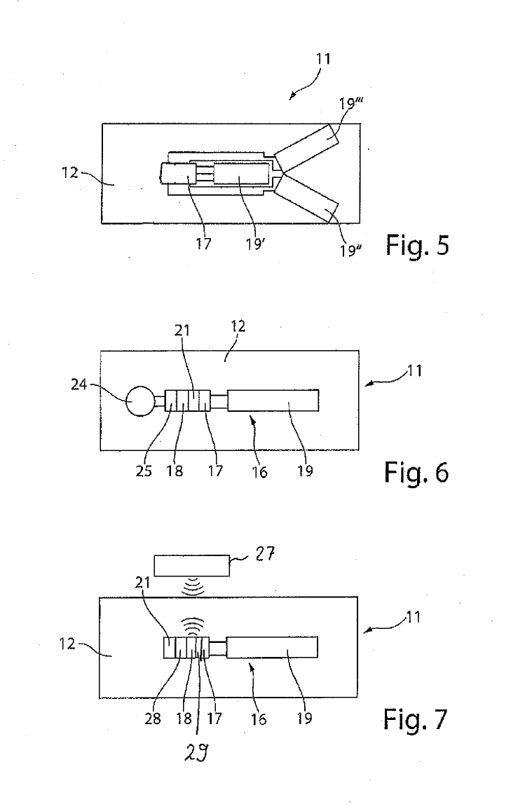

FIG. 5, a schematic view of a further alternative embodiment of the detection device to FIG. 1,

FIG. 6, a schematic top view of a further alternative embodiment of the detection device to FIG. 1 and

FIG. 7, a schematic view of a detection device according to FIG. 1 in communication with a mobile terminal device.

FIG. 1 shows a schematic view from above of the detection device 11. A schematic side view thereof is depicted in FIG. 2. The detection device 11 consists of a carrier element 12, which has an adhesive layer 14 on one side, in order to position the detection device 11 directly on the skin of a user. This adhesive layer 14 is preferably hypoallergenic and covered by a protective film 15, which is removed before the application of the detection device 11 on the skin of the user. Alternatively, several detection devices 11 can be arranged one next to the other on one protective film 15, said detection devices 11 being able to be removed individually therefrom as needed.

A stretch measuring device 16 is applied to the side of the carrier element 12 opposite the adhesive layer 14. This is contacted by a circuit 17, which can be connected to a storage element.

The carrier element 12 is formed tape or strip-like. Preferably, this can consist of a fabric. Alternatively, breathable and/or air-permeable films or a fleece or felt can be provided, which allow for a stretch and/or compression at least in one extension direction.

The stretch measuring device 16 can be provided as an element on the carrier element 12, said element being applied on a film. Alternatively, the stretch measuring device 16 can also be pressed directly onto the carrier element 12. For example, this can be pressed on by a conducted paste, or sprayed on or knife-coated, in order to apply the stretch measuring strip 19 and the corresponding contact points 20, which form the stretch measuring device 16. Adjacent to this, the circuit 17, in particular an IC chip, can be applied. This can, in turn, be connected to a storage element. In order to protect the electronic components, a protective coating 22, in particular a protective lacquer, is preferably applied to the carrier element 12, said protective lacquer preferably completely covering the electric components.

The stretch measuring device 16 described above can, alternatively to the stretch measuring strip 19, also be formed by at least one wire element, which is depicted, for example, in FIG. 1, applied meander-shaped or zig-zagged on the carrier element 11, stitched on and/or even woven into the carrier element 11. As a result of such wire elements, a change of the resistance in the event of a stretch or compression can also be detected and evaluated.

The detection device 11 is formed longer in a first extension direction along the X-axis than in the second extension direction, the Y-axis. This carrier element 12 is suitable for receiving at least one stretching or compressing along the first extension axis, i.e. the X-axis. Correspondingly, the stretch measuring device 16 is also set up relative to the first extension direction. As a result of such a detection device 11, a change in the posture, for example, can be detected, in particular of an ergonomically favourable posture or seating position. For example, this detection device 11 can be aligned along a spinal column on the back, in particular a back muscle, for example applied between the shoulder blades. If an increasing curvature of the back takes place, by leaving an ergonomically favourable seating position because of negligence or tiredness, a stretch in the X direction, which, in this case, corresponds to an extension along the spinal column, takes place via the stretch measuring device 16. Such a change can be detected and optionally stored or displayed.

In FIG. 3, a schematic side view of an alternative embodiment of the detection device 11 of FIGS. 1 and 2 is depicted. The electric components in this embodiment according to FIG. 3 correspond to those of FIGS. 1 and 2. Deviating from this, the stretch measuring device 16 is worked into or embedded in the carrier element 12. For example, the stretch measuring strip 16 in the embodiment of the carrier element 12 as the fabric can be formed as a resistance wire and directly woven into the fabric, also the contact points 19 for connecting the circuit 17. The circuit 17 and, where necessary, the storage unit can be mounted on one of the two sides of the carrier element 12 or, for example, at least partially inserted into a recess in the carrier element 12. If the recess is formed pointing to the adhesive layer 14 and opposite an extensively sealed surface by the carrier element 12, a protective layer 22 is not needed.

In FIG. 4, an alternative embodiment of FIG. 1 is depicted in a top view. In this embodiment, the stretch measuring device 16 is formed from three stretch measuring strips 19 lying one on top of the other as a so-called stretch measuring rosette. The stretch measuring strip 19′ extending in the first extension direction is superimposed on a second and a third stretch measuring strip 19″, 19′″ that are firstly aligned at plus 45° and minus 45° relative to the first extension direction. As a result of such a stretch measuring device 16, changes both in the first and the second extension direction—i.e. in the X and Y direction—and all force directions lying in the XY plane lying in between are detected, evaluated and, where necessary, displayed.

In FIG. 5, an alternative embodiment to FIG. 4 is depicted. This notably deviates in that the three stretch measuring strips 19′, 19″, 19′″ are not provided lying one over the other, but separately on or in the carrier element 12. In terms of the function, this embodiment corresponds to that in FIG. 4.

The embodiments of the three stretch measuring strips 19′, 19″, 19′″ described above, which are applied as pre-manufactured elements, applies analogously for individual wire elements, which are stitched onto the carrier element 12 or woven into the carrier element 12, in terms of the alignment of the stretch measuring strips 19′, 19″, 19′″. With the use of several wire elements, for example, one of the wire elements can be woven into the fabric of the carrier material, and a further wire element can be stitched onto the carrier element 12.

FIG. 6 shows a first embodiment of the detection device 11, which is supplemented by a current accumulator 21, an activation button 24 and a vibration element 25 and/or an acoustic element. This detection device 11 can be used to monitor a posture or a movement sequence independent of further technical aids, and can have a storage element 18.

By means of the activation button 24, the detection device 11 is activated after the application on the body region or the body part and is transferred to the monitoring mode after running through a calibration routine. In the event of a change of the posture or the movement sequence to be monitored, a corresponding signal can be triggered by the vibration element 25 and/or the acoustic element, which communicates the change to the user.

Furthermore, the activation button 24 and/or the circuit 17 can be formed in such a way that the detection device 11 can also be started contactlessly by means of a mobile terminal device 27, by this activating the activation button 24 or the circuit 17. In this embodiment, the detection device 11 can be used both without the use of a mobile terminal device 27 and by using the mobile terminal device 27.

An alternative embodiment of the detection device 11 to FIG. 6 is depicted in FIG. 7. In this embodiment, in addition to the components of the circuit 17, the storage element 18, the data transfer module 28 and/or the energy source 21, an acceleration sensor 29 is provided. A further location and position recognition can be detected by means of this acceleration sensor 29, whereby the monitoring is specified, and further monitoring parameters can be requested.

In this embodiment in FIG. 7, the detection device 11 communicates with a mobile terminal device 27. To do so, the circuit 17, which can also comprise the storage element 18, is coupled to a data transfer device 28, such that the signals detected by the circuit 17 can be contactlessly transferred to the mobile terminal device 27. Similarly, the detection device 11 can be started via the mobile terminal device 11 after the application to body part or the body region. The mobile terminal device 27 can be a smartphone, a tablet, a watch or a further reading device, which is suitable for contactless reading of data. For this purpose, a reading device is also understood that reads an active or passive transponder and/or can communicate with this.

The embodiment described above according to FIGS. 1 to 6 can also be formed facultatively with such an acceleration sensor 29. The embodiments according to FIGS. 1 to 5 can also additionally comprise a contact storage element 18 and/or an energy source 21 and/or an activation button 24 and/or a vibration element 25 and/or a data transfer module 28 and/or an acceleration sensor 29.

Furthermore, it can be provided that, with a central arrangement and application of the stretch measuring device 16, the further electronic components, such as the circuit 17, the storage element 18, the energy source 21, the activation button 24, the vibration element 25, the data transfer module 28 and/or the acceleration sensor 29 can be applied eccentrically, such that there is not an impediment when stretching and/or compressing the carrier material as a result of these components in the axis of the stretch measuring device 16.

With the use of a new detection device, the coupling or the binding of the detection device to the mobile terminal device can take place by means of a code. This code can be provided on the carrier material of the detection device and/or on the packaging of the detection device. Here, such a code can be, for example, a barcode, QR code, number code or similar.

1. A detection device for detecting and monitoring a posture or a movement sequence or a training sequence of a body part having a stretch measuring device, wherein a carrier element is provided that, on one side, has an adhesive layer for adhering to the skin of a user, wherein on or in the carrier material, the stretch measuring device is provided which detects measuring signals in at least one extension direction of the carrier element by a stretch and/or compression of the carrier element, wherein the stretch measuring device is contacted with a circuit formed as a transponder and is fixed on the carrier element, wherein the carrier element is formed tape-like, wherein the carrier element is produced from a fabric, fabric tape, a breathable and/or air permeable film, from a fleece or a felt, wherein the carrier element having a stretch measuring device and the circuit is formed as a disposable product.

2. (canceled)

3. (canceled)

4. (canceled)

5. (canceled)

6. The detection device according to claim 1, wherein the stretch measuring device and/or the circuit and/or the storage element and/or an acceleration sensor are applied to the carrier element, adhered, pressed on, moulded on, knife-coated and/or sprayed on.

7. The detection device according to claim 1, wherein at least the stretch measuring device, the circuit and/or the storage element and/or an acceleration sensor is provided at least partially between the carrier element and the adhesive layer.

8. The detection device according to claim 1, wherein at least the stretch measuring device is woven into or stitched onto the carrier material, and at least the circuit and/or the storage element and/or an acceleration sensor are arranged on one of the two sides of the carrier material or at least partially introduced in a recess of the carrier material.

9. The detection device according to claim 1, wherein the stretch measuring device and/or the circuit and/or the storage element and/or an acceleration sensor are covered by a protective film.

10. The detection device according to claim 1, wherein the stretch measuring device comprises at least one stretch measuring strip or at least one wire element with contact points, wherein two or three stretch measuring strips or wire elements are each formed separately and spatially removed from one another or in the form of a stretch measuring rosette, which are separated from one another via a dielectric material and applied lying one on top of the other, and wherein at least one stretch measuring strip or at least one wire element is woven into the carrier material and/or at least one stretch measuring strip or at least one wire element is applied to the carrier element.

11. (canceled)

12. The detection device according to claim 1, wherein the circuit comprises an electronic control device for evaluating the measuring signals and/or a data transmission device.

13. The detection device according to claim 1, wherein the circuit is contacted with a current accumulator.

14. The detection device according to claim 1, wherein the carrier material has an acoustic element and/or vibration element.

15. The detection device according to claim 1, wherein the carrier material has an activation button for activating the circuit or the circuit can be activated directly by a mobile terminal device or the circuit can be activated via an activation button by means of the mobile terminal device.

16. The detection device according to claim 1, wherein the circuit and preferably the storage element are formed as a passive or active transponder and a contactless data transfer between a mobile terminal device or a reading device and the transponder is provided.

17. (canceled)

18. The detection device for detecting and monitoring a posture or a movement sequence or a training sequence of a body part, wherein a mobile terminal device wirelessly communicates to a data processing program stored thereon or a downloadable data processing program on a mobile terminal device having a detection device according to claim 1.

19. A method for detecting and monitoring a posture or a movement sequence or a training sequence of a body part,
in which a carrier element, which comprises a stretch measuring device and a circuit contacted therewith, is applied directly to a skin of the user by an adhesive layer provided on the carrier element,
in which a calibration routine is started by an activation button on the carrier element and/or on the mobile terminal device,
in which, after the calibration routine to detect the ergonomic posture or the optimum movement sequence or training sequence of a body part, the monitoring of the posture or the movement sequence or the training sequence of the body part is started and a change in posture or the movement sequence to be monitored or the training sequence to be monitored is detected and at least one signal is emitted to the user as indication of the detected change in the posture or the movement sequence or the training sequence of the body part or an actuation signal on adopting or maintaining the posture, the movement sequence or the training sequence.

20. The method according to claim 19, wherein an acoustic and/or vibration signal is emitted by an acoustic and/or vibration element of the carrier element and or the mobile terminal device.

21. The method according to claim 19, wherein the signal is transferred and emitted by a contactless data transfer to a mobile terminal device or wherein the signals received on the mobile terminal device when monitoring the posture or the movement sequence or the training sequence of the body part are stored in an app and emitted.

22. (canceled)

23. The method according to claim 19, wherein a change of posture in the monitoring phase is monitored by an acceleration sensor and a change of the posture to be monitored that takes place only momentarily and returns to the posture to be monitored is recognised and not emitted as an alarm signal.

24. The method according to claim 19, wherein
the calibration routine in a detection device is started by means of an acoustic and/or vibration element by an activation button provided on the carrier element or by the mobile terminal device,
an acoustic and/or vibrating signal is emitted and the user subsequently adopts the posture to be monitored,
after a predetermined time interval or the actuation of the activation button or by the mobile terminal device, a confirmation signal for detecting the posture to be monitored is emitted,
the user adopts at least one first incorrect posture and resulting signals are detected by the circuit and at least one second confirmation signal is emitted after a predetermined time interval or actuation of the activation button or by the mobile terminal device and
subsequently, the calibration routine ends, and the monitoring and/or training mode is started.

25. The method according to claim 19, wherein
the calibration routine in a detection device having an acoustic and/or vibration element is started by an activation button provided on the carrier element or the mobile terminal device,
a first acoustic and/or vibrating signal is emitted, and the user subsequently adopts a starting position for the movement sequence to be monitored or the training sequence,
a confirmation signal is emitted after a predetermined time interval or the actuation of the activation button or the mobile terminal device,
the user carries out the movement sequence or training sequence to be monitored and the body part returns to the starting position and, subsequently, at least one further confirmation signal is emitted after a predetermined time interval or the actuation of the activation button or the mobile terminal device and
subsequently, the calibration routine ends, and the monitoring and/or training mode is started.

26. The method according to claim 19, wherein
the calibration routine in a detection device having a contactless data communication to a mobile terminal device is started by the mobile terminal device,
the user adopts the posture to be monitored, and, after a predetermined time duration or by confirmation of the user on the mobile terminal device, this posture is detected and stored,
the user adopts at least one incorrect posture and the resulting signals are detected and at least one confirmation signal for detection is emitted and a confirmation is input by the user and
subsequently, the calibration routine ends, and the monitoring and/or training mode is started.

27. The method according to claim 19, wherein
the calibration routine in a detection device having a contactless data communication to a mobile terminal device is started by the mobile terminal device,
the user adopts a starting position for the movement sequence or training sequence to be monitored, and, after a predetermined time duration or by confirmation on the mobile terminal device of the user, this starting position is detected,
the user carries out the movement sequence or training sequence to be monitored and the body part returns to the starting position and at least one confirmation signal is emitted by the terminal device or the starting position is confirmed by the user, and
subsequently, the calibration routine ends, and the monitoring or training mode is started.
